(19) United States
(12) Patent Application Publication
(10) Pub. No.: US 2006/0204480 A1
(43) Pub. Date: Sep. 14, 2006

(54) Replication competent virus or its recombinant for use in cancer therapy

(76) Inventor: Ari Hinkkanen, Turku (FI)

Correspondence Address: JAMES C. LYDON, 100 DAINGERFIELD ROAD, SUITE 100, ALEXANDRIA, VA, 22314, US

(21) Appl. No.: 11/435,889

(22) Filed: May 18, 2006

Related U.S. Application Data
(63) Continuation-in-part of application No. PCT/FI05/00230, filed on May 19, 2005.

(30) Foreign Application Priority Data
May 21, 2004 (FI) 20040704

(51) Int. Cl. A61K 48/00; C12N 15/86

(52) U.S. Cl. 424/93.2; 435/456

(57) ABSTRACT

A method for treatment of a central nervous system cancer in an individual which includes administration, preferably peripheral administration, of a replication competent alphavirus of the Semliki Forest family, or its recombinant, to the individual.

This invention relates to the use of a replication competent alphavirus or its recombinant in cancer therapy.

### BACKGROUND

Malignant central nervous system cancers, such as gliomas, are mostly treated using surgery or radio- and chemotherapy. Recently, a virus-mediated gene therapy has emerged as one of the interesting alternatives, and vectors based on different virus types have been developed for this purpose. Use of oncolytic virus vectors in the treatment of fresh brain tumors is proving to be a very promising technique (Shah et al. 2003). Although, for safety reasons, the vast majority of research has focused on the use of viruses which are incapable of reproduction (replicons), at the same time, research has been continued using viruses which are able to divide (replication competent), but which have been genetically attenuated, weakened, and are as harmless to the host organism as possible.

The potential of replication competent viruses to destroy tumor cells is multiple, but targeting them is difficult. Only recently have replication competent herpes simplex, adeno and rubella viruses been used in tumor targeting. Use of the herpes simplex virus in the treatment of glioma has already proceeded to phase 2 trials (Markert et al. 2000, Georger et al. 2003), and adenoviruses are already being tested in the treatment of different cancers (Khuri et al. 2000). A replication competent virus may be, for example, a pure natural virus, or its variant, which has preserved its ability to infect and reproduce in a tissue for days or months. These viruses, which incorporate the envelope of natural viruses, but from which some of the genes have been removed or modified, so that the virus can not produce new viruses in tissues, are called replicons. These viruses have been artificially produced in cell lines which help the artificially attenuated gene machinery of the virus to generate a virus which is able to infect one single time, but formation of new viruses is impossible due to the defective virus genes.

Virus-mediated elimination of tumor cells is affected, on one hand, by lysis generated by virus replication, and on the other hand by a therapeutic gene which restricts cell reproduction. Cell destruction in an immunocompetent organism increases an immune response towards the tumor. The ability of tumor cells to stimulate the immune cells which are destroying them increases if these cells are made to produce certain cytokines. A tumor can be manipulated to secrete a cytokine by infecting tumor cells with a recombinant virus which produces a cytokine. For example, interleukin-4 (IL-4) and IL-10 and IL-12 cytokines have been used as tumor vaccines in therapeutic trials (Elder et al. 1996; Andreansky et al. 1998; Melani et al. 1994; Colmenero et al. 2002; Okada et al. 2003). Also, angiogenesis-inhibitors have been tested using virus mediation. For example, protease inhibitors have been used in order to prevent vein formation in human melanomas with the help of adenoviruses (Ahonen et al. 1998, 2002, 2003). Cell destruction can be affected not only through the immune system, but also by directly influencing the reactions controlling the apoptosis. The cytokine TGF-B (transforming growth factor) has been shown to cause apoptosis of hepatocarcinoma cells (Chabicovsky et al. 2003), but on the other hand, it has also been observed to promote mobility of cancer cells (Wick et al. 2001). Moreover, nerve growth factors have been observed to be able to control the growth of glioma cells (Yaeger et al. 1992; Kimura et al. 2002). In addition, a thymidine kinase gene (TK) attached to the therapeutic virus produces an enzyme which converts a harmless medicine in a (tumor) cell infected by the virus into a cytotoxin.

Alphaviruses have been used in a tumor therapy with a mediocre success, but these have been replicon-based viruses (Colmenero et al. 2002). The Semliki Forest virus (SFV) is a murine pathogen, which, however, infects human cells, and every now and then SFV causes symptomatically mild epidemics in different regions in Africa. The Venezuelan equine encephalitis virus VEE, which causes severe meningitis, has been used to develop vaccine strains for vaccinating humans and livestock. The wild type of SFV virus is lethal to adult mice, but its attenuated strain, SFV A774, does not cause symptoms in adult mice. The gene areas affecting SFV virulence have been recently identified, and they were discovered to be located in several places in the area of the replicase genes (Tuittila et al. 2000, 2003).

Patent publications US-2003-0232035 and WO 96/21416 describe extensively the use of different recombinants of alphaviruses in therapy, for example, in treatment of different tumors. These publications do not, however, handle the use of live virus recombinants, but replicons which are incapable of reproduction. In replicon systems, a virus is genetically defective in such a way that having infected once, it can not reproduce new viruses in cells (Liljeström et al. 1991. J Virol 65, 4107-4113). Replicon viruses have been developed for SFV, the Sindbis and VEE-viruses, and some of them are commercially available.

Treatment of gliomas is mostly a combination of conventional chemo- and irradiation therapies after surgical removal of the tumor. Surgery, although necessary and life-saving, does not effectively remove the entire tumor mass and tumors tend to rapidly regrow. This is partly because it is difficult to define the borders between tumor and the healthy tissue which is responsible for vital body functions. It is therefore of importance to be able to treat the residual malignant cells after the operation has been completed. Irradiation therapy and chemotherapy are a burden to the glioma patient compromising the important immune functions to control tumor growth. Therefore it would be advantageous if viral therapy could be applied, where the virus is not directly introduced into the operation wound but is introduced peripherally, some time later from the operation, to allow the patient to recover from surgery. Such therapy could be achieved with neurotropic virus which has retained its capability to replicate but shows highly attenuated phenotype, similar to live vaccine viruses. Another important feature of such therapy virus would be that it preferentially infects CNS glial cells instead of neurons with highly vital functions, and that viral replication is restricted in whole CNS.

The A774 strain of Semliki Forest virus has been isolated from a pool of mosquitos in Mozambiq 1957 and was derived from a SFV stock isolate based on its low virulence in mice. The A774 virus has evolved separately from wild type SFV and has never gained in virulence in laboratory conditions. Virulence determinants are spread over the non-structural genome region and reversion to virulence is highly improbable (Tuittila et al. 2000, Tuittila & Hinkkanen 2003). The virus is neurotropic and when administered via peripheral routes, e.g. the peritoneal cavity, via blood stream, into muscle or via the respiratory track, the virus finds its way to the CNS. However, the infection is focal and limited to the perivascular space with viral titers 100-1000 fold lower compared to wild type SFV. This makes A774 virus highly non-pathogenic to mice, causing blood and CNS viremia, but no symptoms or clinical disease are observed. Transient myelin loss can be observed but the mice remain clinically healthy. It is well known that SFV also infects humans. In certain areas of Africa marked portion of population is seropositive but there is no disease associated with SFV infection. However, there are indications that SFV reaches the CNS as in acute infections the symptoms include headache, nausea and fever, all typical signs for mild CNS infection (Mathiot et al.).

### SUMMARY OF THE INVENTION

On one hand, this invention is based on the observation that the virus vectors designed for preventing inflammation infect and destroy different types of tumor cells extremely efficiently (Tuittila et al. 2000, Vähä-Koskela et al. 2003; Ehrengruber et al. 2003). For example, human A2058 melanoma and rat L9 gliocarcinoma cells died in 48 hours when using the virus vector VA 7. On the other hand, the invention is based on the observation that the SFV A774 strain reproduces highly efficiently in developing cells of the central nervous system (mice under 2 weeks), whereas reproduction is very limited in neurons or glia cells of an adult mouse (<1000×) (Amor et al. J Gen Virol. 1996; 77:281-91). The virus in question is not pathogenic to adult animals, and therefore the inventors decided to test, whether the intravenously administered virus would find its way to tumor cells, and especially in a tumor located in the brains, which is otherwise a difficult area to reach. In an experiment presented below—which concerned human A2058 melanoma cells in an SCID/SCID mouse model—it became clear that the hypothesis presented by the inventors was correct.

A non-pathogenic strain of the Semliki Forest virus, SFV A774 (abbr. A774), reproduces in tumor cell lines better than, for example, in the brain tissue itself of an adult mouse. SFV infects human cells, and Africa has experienced SFV epidemics locally. Thus, SFV also causes infections in humans, but not any disease. SFV A774 hardly reproduces at all in the brain cells of mice, rather it reproduces profusely in tumor cells (for example, brain tumor, glioma, and gliosarcoma). Therefore, the inventors developed an idea to use the A774 strain (or another avirulent SFV strain or avirulent alphavirus strain) for treating a human tumor, especially a brain tumor, optionally alongside with other forms of therapy.

According to one aspect, this invention concerns a method for the treatment of a central nervous system cancer in an individual, said method comprising peripheral administration of a replication competent alphavirus of the Semliki Forest family, or its recombinant, to said individual.

According to another aspect, the invention concerns a method for the treatment of a central nervous system cancer in an individual, said method comprising a combination of a surgical operation of a tumor in the central nervous system and administration of a replication competent alphavirus of the Semliki Forest family, or its recombinant, to said individual.

According to a third aspect, the invention concerns a pharmaceutical composition comprising a replication competent alphavirus of the Semliki Forest family, or its recombinant, and a therapeutically suitable carrier.

### BRIEF DESCRIPTION OF THE FIGURES

FIG. 1 shows the effect of a replication competent SFV VA7-EGFP recombinant virus (derived from SFV A774) on the volume of the human melanoma tumor A2058 in a SCID/SCID mouse model. The tumor volume is presented as a function of time according to the different administration methods (i.v.; i.t.; i.p.) of the recombinant virus and of the control (PBS). Each bar represents an average of 3 mice.

FIG. 2 shows illustratively the effect of the above mentioned replication competent SFV VA7-EGFP recombinant virus on the tumor size in a SCID/SCID mouse after 3 weeks of virus administration (i.v.; i.t.; i.p.), and in the control (PBS).

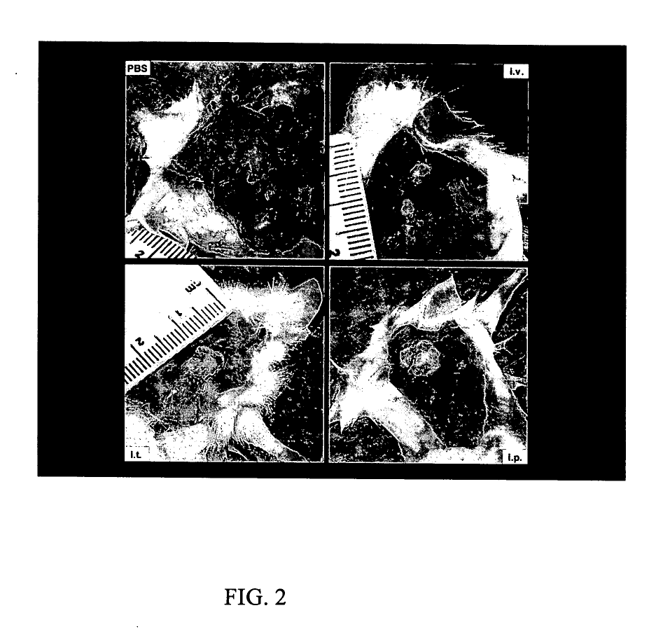

### DETAILED DESCRIPTION OF THE INVENTION

A replication competent virus may be, for example, a pure natural virus, or its variant, which has preserved its ability to infect and reproduce in a tissue for days or months, whereas viruses which incorporate the envelopes of natural viruses, but from which some genes have been removed or modified, so that having infected once, the virus can not produce new viruses in tissues, are called replicons. These viruses have been generated artificially in cell lines which help the artificially attenuated gene machinery of the virus to reproduce virus particles capable of a single infection, but the formation of new viruses is impossible because only incomplete genome is incorporated into replicon viral particles. Normal tissue and cells do not make the reproduction of replicons possible, although the infection takes place once in order to manifest the therapeutic gene. A virus, which has been achieved by modifying its envelope proteins so that it is weakened but still can reproduce, is not a replicon but a replication competent virus.

The use of a replication competent alphavirus, or its recombinant, can be adapted for the treatment of any type of cancer. This therapeutic method is especially well-suited, however, for the treatment of central nervous system cancers, for example, brain cancer or gliomas.

The said alphavirus can be any reproducible alphavirus which can be used safely. In treatment of central nervous system tumors, it is recommended that the reproduction of the selected alphavirus be very limited, or it should not reproduce at all in the normal central nervous system cells (for example vaccine strains) of an adult individual.

A preferably selected alphavirus is capable of finding its way to central nervous system cells when administered intravenously.

An alphavirus can aptly be a virus of the Semliki Forest virus family. Especially recommended is the Semliki Forest strain A774 which has been proven to be very safe.

In adult mice the replication of A774 strain is highly restricted, but in neonatal mice younger that two weeks it causes fulminant viremia and death of the animals (Amor et al. J Gen Virol. 1996; 77:281-91). We have data suggesting that this is due to low relative expression of anti-inflammatory cytokines in neonatal brain. A774 is highly specific for glial cells and only occasionally neurons are infected. This is of great importance when virus is given in therapy purposes as to save the vital functions of neurons. As, for example, SFV replicon is based on WT SFV (known to be able to infect and replicate in neurons), infections are targeted to neurons with this virus. Glial cells predominate in the brain but do not represent as numerous functional subgroups as neurons do. Thus, even if only a small fraction of glial cells gets infected, the consequences are probably milder that in infection of neurons.

The entry into CNS of SFV most probably occurs through infection of brain capillary endothelial cells and thus the first barrier on the way to intracranial tumors is the capillary endothelium. Entering by virus to brain through the blood brain barrier (BBB) necessitates either BBB damage, or the virus should have potential to overcome the barrier in order to home to CNS. A774 is capable of penetrating BBB and can reach brain parenchyma, and importantly, its ability to replicate allows its escape from the first barrier cell layer into the intern of CNS. This is not the case with so called replicons as they only can infect one cell layer after which they become unable to form progeny virus. Thus BBB endothelial cells will filter replicon type viruses out of blood stream and prevent them from reaching the tumor. In contrast, A774 penetrates the barrier and by replicating effectively in different types of tumor cells it most probably will be able to find into the tumor tissue and start to replicate leaving, however, adult healthy CNS tissue untouched due to its restriction in replication.

Other suitable alphaviruses worth mentioning are Venezuelan equine encephalomyelitis (VEE), Western equine encephalomyelitis (WEE), and Eastern equine encephalomyelitis (EEE) and Sindbis virus.

An alphavirus can be used as such or as its recombinant. Recombinants make it possible to transport a foreign gene, which has an advantageous effect on the treatment of cancer, to the tumor. The desired therapeutic gene is cloned to the virus in a gene-technologically determined cloning site, for example, for the control of the virus's own subgenomic 26S promoter. A vector thus obtained can be used to produce a recombinant virus RNA in vitro, which is then transported to suitable host cells using electroporation or lipofection. The cells start producing the recombinant virus which is harvested. Suitable inserts, therapeutic genes, may be, for example, thymidine kinase of Herpes simplex virus, interferon(IFN)-gamma, IL-12, IL-4, TGF-beta TNF-alpha, IL-2, TIMP 1-4, or epitopes of a T-cell activator such as lymphotoxin (LT), which activates tumor-specific T-cells. A therapeutic gene may also be a small interfering RNA (siRNA) which is built to inhibit genes that promote cell division, and/or to activate cell death.

To obtain the virus, cDNA which codes the therapeutic gene, is cloned into the RNA expression vector which is used to produce the recombinant virus genome in the RNA form. The RNA in question is infective and is capable of initiating a virus protein synthesis in eucaryotic cells. New virus particles are formed in cells, and the viruses are reproducible and manifest the therapeutic gene if such a gene has been cloned to the virus genome. The produced virus is added in BHK hamster cells and the virus is titred using a crystal violet method. Virus stocks are stored in −70° C.

In animal tests, mice (rats) are infected intraperitoneally, intratumorally or intravenously using a suitable amount of virus, for example, 1 million infective particles.

In clinical use, the alphavirus, or its recombinant, is preferably administered to a patient peripherally (systemically), for example, intravenously, intra-arterially, intramuscularly, intraperitoneally, subcutaneously, intradermally or intranasally. Alternatively, if the alphavirus or its recombinant is used in combination with surgical operations, the alphavirus or its recombinant can be administered locally, for example intratumorally or intracranially. The selected administration method, dosage amount and dosage regimen depend especially on the nature and location of the tumor, and also on the alphavirus or its recombinant that is used.

Here we propose the use of SFV A774 alone or in combination with tumor surgery to abrogate residual malignant cells from the tumor cavity by administration of the virus via alternative routes of which the following routes are mentioned as examples only:

1. Intraperitoneally, i.e. the virus is injected in PBS into the peritoneal cavity from where it first spreads to the blood stream causing blood viremia, but simultaneously it begins to penetrate the BBB into the brain. To reach the CNS takes 2-3 days in mouse.

2. Intravenously, i.e. virus is injected directly into the blood circulation, whereby the dose probably can be lower. Also in this case the virus infects blood cells but simultaneously starts to penetrate the BBB into brain parenchyma. From blood the virus disappears by day 3 post-infection in mice. Brain viremia durates about five days. This is dependent on immune response which varies between mouse strains and human individuals.

3. Intramuscularly, i.e. virus is injected into muscle, e.g. the thigh muscle from where it is transferred via blood stream to brain capillaries and enters the CNS.

Generally, A774 is very ineffectively infecting peripheral organs, it is hardly detectable with immunohistochemistry but can be found in virus titration in very low numbers in liver, kidney and muscle (our unpublished observations).

4. Intranasally, i.e. the virus is given as an aerosol via nasal track followed by entry via mucosal tissue. In mice very small numbers of virus in small volumes can be given i.n. leading to effective CNS infection.

5. Intracranially, i.e. via directly injecting into or smearing the virus on the tumor cavity walls. This approach, however, should only be done in combination with operations.

Emphasis is laid on the properties of A774 to protect the neurons while infecting glial cells in a limited fashion. It is highly likely that the vasculature of the tumor is more fragile compared to intact BBB, and hence the restriction for virus entry is less stringent. Given via routes described above virus most probably enters tumor tissue more effectively compared to intact CNS tissue.

Administration of replicative, nonpathogenic virus via peripheral routes described above would allow the immune system to retain its tumor reactivity, which is not the case e.g. in radiation therapy. Virus therapy could be the first form of complementing therapy aiding the surgical removal of the tumor body. In addition, destruction of tumor cells by viral infection probably also enhances cytotoxic immunity against the tumor-specific antigens thus speeding up the removal of tumor. This is especially true as the virus will function as an adjuvant by first replicating in the peripheral organs (blood, lymph nodes, spleen) producing antiviral cytokines such as interferons.

The virus is administered in a pharmaceutical composition, which is advantageously a solution, microemulsion or nanoemulsion in a phosphate-buffered saline solution, or in a corresponding physiological saline solution, where the virus stays unharmed. The virus can also be mixed with a carbohydrate gel, or corresponding polymer containing saline or the like.

The time of virus delivery can be prolonged by inserting the virus in small beads of suitable biomaterial (polymer, porous, semipermeable) which contains either cells in culture medium harbouring the virus, or pure virus in gel-like medium, which slowly releases the virus to prolong the viremia. From immunological point of view use of SFV should be effective in those geographic areas where antibodies to alphaviruses are rare.

The invention will be described in more detail in the following non-limiting experimental section.

### EXPERIMENTS

The purpose of the following experiments is to determine, whether it is possible to use the SFV A774 strain, the VA7 vector virus produced from it, or its variants (Vähä-Koskela et al. 2003; Ehrenburger et al. 2003), to destroy a tumor with intratumoral infection, or as an oncolytic vector directed towards the tumor. Human melanoma, rat gliosarcoma and mouse glioma cells are used as models. Both the SFV A774 parental virus and the VA7 vector seek their way to the central nervous system, but their replication is limited in the brain tissue of an adult mouse, whereas in young mice, the A774 strain reproduces profusely in central nervous system cells. The replication environment of tumor cells may resemble the condition of a developing nervous system, since their innate immune response may be weakened, in which case the A774 virus is able to reproduce more efficiently in these cells, at least in cell cultures. The research determines whether the A774 seeks the tumor as efficiently as it seeks other tissue—or more efficiently—and whether the virus replicates in the tumor, and whether the replication causes oncolysis without notably damaging the surrounding healthy cells (Sauthoffet al. 2003). A study will also be done to find out whether it is possible to promote the destruction of a tumor by using a therapeutic gene attached to the VA7 vector. A group of recombinant viruses (Vähä-Koskela et al. 2003; Ehrenburger et al. 2003) has been produced in association with an earlier myelin study, and the following SFV A774-based constructs, in which the VA7 vector virus has been used, and of which the majority also includes a therapeutic gene suitable for tumor therapy, are ready: VA7-interleukin (IL)-4 (Krauss et al. 1995), VA7-IL-10 (Kohno et al. 2003), VA7 tissue inhibitor of metalloprotease (VA7-TIMP)-1,-2,-3 (Ahonen et al. 2002; Spurbeck et al. 2003; Takahashi et al. 2002), VA7-HSV thymidine kinase (VA7-HSV-TK) (Loimas et al. 2001), VA7-nerve growth factor (VA7-NGF) (Kimura et al. 1992), VA7 brain-derived neurotrophic factor (VA7-BDNF), VA7-transforming growth factor (VA7-TGF)-beta (Ramont et al. 2003), the vector SFV VA7 itself, and VA7 EGFP (Vähä-Koskela et al. 2003). In order to study the oncolytic efficiency, the VA7-EGFP vector which produces a fluorescent marker protein is used at first. This study is useful when evaluating the proportion of the oncolytic effect of the SFV-VA7vector in the synergism of the therapeutic gene and oncolysis.

### EXAMPLE 1

### Effect of a Replication Competent SFV VA7-EGFP Recombinant Virus on Human A2058 Melanoma Cells in a SCID/SCID Mouse Model

A replication competent SFV VA7-EGFP (enhanced green fluorescent protein, from jelly fish, manet) efficiently infects A2508 melanoma cells and rat 9L cells in vitro, and the cells die nearly 100% within 48 hours (Vähä-Koskela et al. 2003). SFV A774 also infects human lung cancer and colon cancer cells, including different malignant cells derived from the central nervous system of a mouse, such as oligodendroglioma, glioblastoma, and mouse melanoma cells. In this pilot experiment, A2058 melanoma cells (1 million) were injected into SCID/SCID (severe combined immune deficiency) mice (16 mice) intradermally (i.d.) in the nape of the neck, and the tumors were allowed to develop for one week, after which the tumors were 5-7 mm in diameter. The mice were anesthetized, and 7 days after the tumor inoculation, they were treated as follows: the SFV VA7-EGFP virus (10(7) pfu/ml: (replication competent, Vähä-Koskela et al. 2003) was injected in animals in doses, in total 100 μl in a PBS solution (PBS phosphate buffered saline); in three mice, 1 million particles of the VA7-EGFP virus was injected into the tumor, in three other mice the same amount was injected into the tail vein, and, in addition, in three mice, 1 million virus particles were injected into the abdominal cavity. 100 μl BPS was injected intravenously (i.v.) into the control mice (3). The mice were monitored for 4 weeks; they were weighed and the tumors were measured. The majority of the mice appeared to be normal for the first three weeks, although they had lost weight. Neurological symptoms manifested in several mice at 3 weeks and thereafter, and this was probably caused by the persistent virus, since the immunodeficient SCID mice are not able to eliminate the virus. Several tissue types (brains, spinal cord, blood, liver, muscle, kidney, heart, spleen, lungs) were taken from the mice for histological tests.

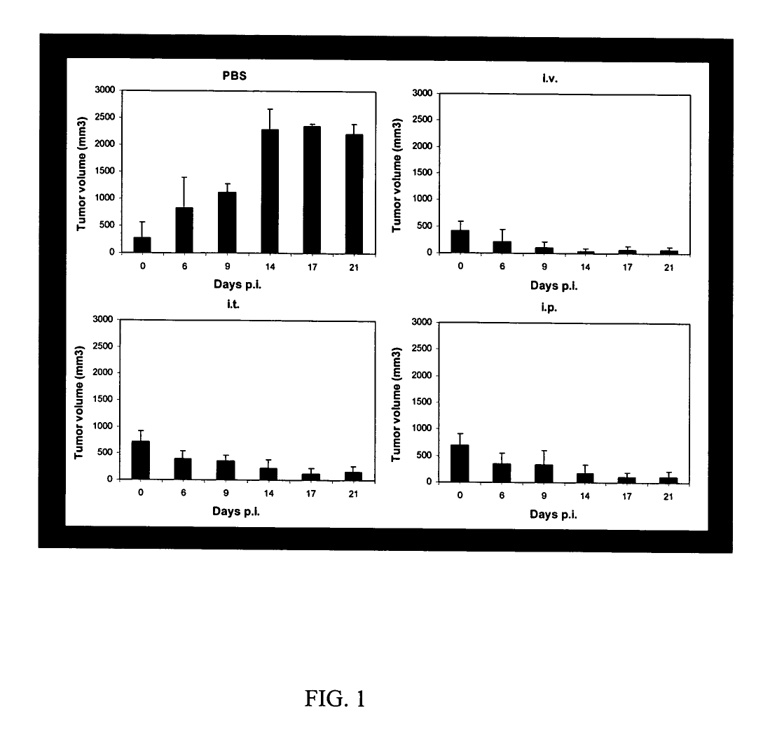

Four weeks after the tumor cell injection, the mice were terminated, and the tumor tissue or the remaining tissues, as above, were collected. At the end of the experiment, some of the mice had neurological symptoms which were caused by the SCID mice'simmunodeficiency, which allowed long-term infection in the spinal cord, damages to the motoneurons, among other things. FIG. 1 (20 days after infection) shows the results of the infection of the tumor mice. On the left, there is a PBS control, the tumor of which had grown considerably (about 2500 mg), whereas in all the mice which had received the virus, the tumor had diminished remarkably (about 50-200 mg). Some of the mice receiving the virus had lost weight considerably, which may be partly caused by viremia, but also by the change caused in their general condition by the tumor, because the PBS control mice also lost weight. The virus injected in the tumor, administered intravenously and in the abdominal cavity, were all very efficient, because in all treated mice, the tumors diminished remarkably (about 50×). The tumor growth was already clearly inhibited one week after the infection.

Histological and in situ hybridization analyses showed that the virus had found its way to the tumor with all administration methods (i.p.; i.t.; i.v.). The inner parts of the tumors were mostly necrotic with no clear morphology. Only single isolated infected (muscle) cells were observed around the tumor. No virus could be found in a healthy brain tissue. Fiber structures formed from the mouse's collagen tissue were observed in the inner parts of the tumors and they appeared to prevent the virus from spreading, since this tissue was not at all infected by the virus. The VA7-EGFP caused an apoptotic cell death, i.e. programmed cell death, at least in some of the tumor cells, and other cells died necrotically.

### EXAMPLE 2

### Effect of SFV Viruses on the Growth of the Rat 9L Gliosarcoma and Mouse Glioma

In order to construct and study suitable animal models, first different cell lines are infected and their survival is then monitored in cell cultures. The cells are infected and the mechanisms of cell death are evaluated. Cell cultures are monitored using a microscope and the cells are dyed at different stages in order to ascertain apoptosis and necrosis. The virus VA7-EGFP (EGFP, enhanced green fluorescent protein) is used as a control, the insert alone of which does not initiate cell destruction. Moreover, the effect of the amount of the infective virus on cell destruction is measured. Different tumor cell types are tested in order to create and analyze new models.

In the brain tumor (gliosarcoma) model, the test animals are female Fischer 344 rats, in which 9L gliosarcoma cells cause subcutaneous or intracranial tumors. The said tumor model is very well known, and it has been developed using just this rat strain. The 9L-glioma cells to be used in the model have been tested in vitro, and, for example, VA7-EGFP and VA7-HSV-TK gene vectors spread in 9L-cells throughout the whole culture in 24 hours and destroy all cells in 48 hours (unpublished and Vähä-Koskela et al., 2003).

Using subcutaneous tumors, rat trials are used to determine whether the SFV A774-based vector (for example VA7-EGFP, SFV-HSV-TK) injected into the tumor is capable of infecting and eliminating the tumor. The tumors are induced in rats and when the tumors have reached a diameter of 1 cm, the virus vector is injected 1×10⁶ viruses per tumor. The animals are terminated 1 day, 2 days, 3 days, 7 days and 21 days after the gene transfer (infection). The tumor and other tissues (blood, tissues surrounding the tumor, spinal cord, liver, heart, lungs, spleen, brains) of the animal are analyzed using immunohistochemistry to find viruspositive and transgene positive cells. If it is shown that the virus is able to transport the therapeutic gene efficiently into the tumor cells (over 20% infected cells), and the virus does not essentially infect healthy tissues and cause cell destruction, a therapeutic test is conducted using the ganciclovir medicine. Enzyme TK (thymidine kinase) of the virus VA7-HSV-TK is powerless as long as there is no ganciclovir medicine in the animal cells. As soon as the virus infects the cell, it starts producing the TK enzyme, and the medicine becomes toxic to the cells, and the cells die. The medicine is harmless in non-infected cells. Since this RNA virus has a very short life span, administration of the medicine is begun prior to that of the virus infection. Virus dosages can be multiplied, escalated and the dosage regimen multiplied (for example every other day 3-5 times a day 1 million viruses). This is because the virus is destroyed efficiently in animals with a normal immune system, but the SFV virus can apparently be injected several times without a major abrogating immune response. Tumor growth is monitored by measuring (using luminescence and/or magnetic imaging) the tumors regularly, and the general condition, and health of the animals are monitored. After the animals have been terminated, the tumors are weighed and the tissues are analyzed histologically to ascertain the presence of the virus and the TK enzyme.

Trials using intracranial tumors will be initiated based on the information obtained from the above mentioned subcutaneous tumor experiments. At first, the study will focus on the route by which the VA7-vector should be administered so that it would be as efficient as possible and would cause as few side effects as possible. At this stage, no ganciclovir will be used, but only the effect of the virus on the animal's life span will be determined. Brain tumors will be induced stereotactically by injecting 9L cells into the brains, and after they have matured, a gene transfer using a virus infection will be performed. The vector will be injected directly into the tumor by using the virus amount which was discovered to be the best in the subcutaneous experiments, or the vector will be injected into the tail vein or the abdominal cavity. Later, the route the virus will take to the tumor when it is inhaled through the nose will be tested. The dosage times and number of single doses of the virus can be increased. Brain tumors are measured using magnetic imaging. The animals will be terminated 1 day, 2 days, 3 days, 7 days and 20 days after the infection. The virus-positive cells and cells containing the therapeutic gene will be analyzed from the tumor and surrounding brain tissue using immunohistochemistry. Histological methods will be used to determine whether the healthy brain tissue was damaged.

At the next stage, a therapeutic trial with ganciclovir (GCV) will be conducted for intracranial tumors using the vector administration method which was discovered to be best for transferring the therapeutic gene to the target tissue. The administration of the ganciclovir will be started before that of the infection at a time specified in earlier experiments. This will ensure that the virus, which lives in the animal only for a couple of days, at most 2 weeks, will transport the gene into the cells, which already contain the ganciclovir medicine. Ganciclovir, however, will also be administered for 10-15 days after the infection, after which the animals will be monitored for 3 more weeks. Tumor growth will be monitored using magnetic imaging and comparing the treatment group (VA7-HSV-TK virus+GCV) to the control group (1. no VA7-HSV-TK virus, GCV, 2. no GCV, VA7-HSV-TK virus).

The survival of animals in different groups will be monitored. When the animals die, either by termination or because the monitoring period has ended, the animals will be analyzed by measuring the tumor size and by studying the brain tissue at the tumor location and at other areas of the central nervous system, and, in addition, by analyzing the tissues of the peripheric organs. Based on this innovation, we are involved in a joint project with the University of Kuopio, which has magnetic imaging equipment for small animals.

Slightly different animal models can be constructed in order to test different types of brain tumors. For example, malignant oligodendrocytes of mice which produce myelin can be planted both subcutaneously and intracranially into the Balb/c mouse strain and the efficiency of the virus therapy will be studied using the VA7-HSV-TK virus and ganciclovir treatment. The effect (VA7-TIMP, see above) of proteolytic enzyme inhibitors (TIMP) preventing vein formation will also be studied. Balb/c mice have a normal immune system, and thus we can test how the therapeutic virus is eliminated from the body. At the same time that we measure the destruction of tumor cells, we measure the infection of normal oligodendrocytes outside the brain tumors. Using a similar test series, we will study the behavior of G261 glioma cells in the C57 black/6 mouse strain with the SFV-VA7-TK infection, with and without the ganciclovir treatment. Spreading of the virus will be measured using titer and histological methods. The clinical condition of the animals will be monitored daily.

The embodiments of the invention described here are illustrative by nature and shall not be limiting to the invention. For experts in the field, it is clear that there are other ways which do not deviate from the purpose of this invention.

### REFERENCES

Ahonen M, Poukkula M, Baker A H, Kashiwagi M, Nagase H, Eriksson J E, Kahari V M. Tissue inhibitor of metalloproteinases-3 induces apoptosis in melanoma cells by stabilization of death receptors. Oncogene. 2003 Apr. 10;22(14):2121-34.
Ahonen M, Ala-Aho R, Baker A H, George S J, Grenman R, Saarialho-Kere U, Kahari V M. Antitumor activity and bystander effect of adenovirally delivered tissue inhibitor of metalloproteinases-3. Mol Ther. 2002 June;5(6):705-15.
Ahonen M, Baker A H, Kahari V M. Adenovirus-mediated gene delivery of tissue inhibitor of metalloproteinases-3 inhibits invasion and induces apoptosis in melanoma cells. Cancer Res. 1998 Jun. 1;58(11):2310-5.
Amor S, Scallan M F, Morris M M, Dyson H, Fazakerley J K. Role of immune responses in protection and pathogenesis during Semliki Forest virus encephalitis. J Gen Virol. 1996 February;77 (Pt 2):281-91.
Andreansky S, He B, van Cott J, McGhee J, Markert J M, Gillespie G Y, Roizman B, Whitley R J. Treatment of intracranial gliomas in immunocompetent mice using herpes simplex viruses that express murine interleukins. Gene Ther. 1998 January;5(1):121-30.
Chabicovsky M, Wastl U, Taper H, Grasl-Kraupp B, Schulte-Hermann R, Bursch W. Induction of apoptosis in mouse liver adenoma and carcinoma in vivo by transforming growth factor-betal. J Cancer Res Clin Oncol. 2003 September;129:536-42.
Colmenero P, Chen M, Castanos-Velez E, Liljeström P, Jondal M Immunotherapy with recombinant SFV-replicons expressing the P815A tumor antigen or IL-12 induces tumor regression. Int J Cancer. 2002 Apr. 1;98(4):554-60.
Ehrengruber M U, Renggli M, Raineteau O, Hennou S, Vähä-Koskela M J, Hinkkanen A E, Lundstrom K. Semliki Forest virus A7(74) transduces hippocampal neurons and glial cells in a temperature-dependent dual manner. J Neurovirol. 2003 February;9(1):16-28.
Elder, E. M., Lotze, M. T., and Whiteside, T. L. 1996. Successful culture and selection of cytokine gene-modified human dermal fibroblasts for the biologic therapy of patients with cancer. Hum. Gene Ther. 7: 479-487.
Geoerger B, Grill J, Opolon P, Morizet J, Aubert G, Lecluse Y, van Beusechem V W, Gerritsen W R, Kim D H, Vassal G. Potentiation of radiation therapy by the oncolytic adenovirus dl1520 (ONYX-015) in human malignant glioma xenografts. Br J Cancer. 2003, 89(3):577-84.
Khuri F R, Nemunaitis J, Ganly I, Arseneau J, Tannock I F, Romel L, Gore M, Ironside J, MacDougall R H, Heise C, Randlev B, Gillenwater A M, Bruso P, Kaye S B, Hong W K, Kim D H. A controlled trial of intratumoral ONYX-015, a selectively-replicating adenovirus, in combination with cisplatin and 5-fluorouracil in patients with recurrent head and neck cancer. Nat Med. 2000 August;6(8):879-85.
Kimura S, Yoshino A, Katayama Y, Watanabe T, Fukushima T. Growth control of C6 glioma in vivo by nerve growth factor. J Neurooncol. 2002 September;59(3):199-205.
Kohno T, Mizukami H, Suzuki M, Saga Y, Takei Y, Shimpo M, Matsushita T, Okada T, Hanazono Y, Kume A, Sato I, Ozawa K. Interleukin-10-mediated inhibition of angiogenesis and tumor growth in mice bearing VEGF-producing ovarian cancer. Cancer Res. 2003 Aug. 15;63(16):5091-4.
Krauss, J. C., Cameron, M. J., Park, A. N., Forslund, K., and Chang, A. E. 1995. Efficient transduction of early passage human melanoma to secrete IL-4. J. Immunol. Methods 183: 239-250.
Liljeström P, Lusa S, Huylebroeck D, Garoff H. 1991. In vitro mutagenesis of a full-length cDNA clone of Semliki Forest virus: the small 6000-molecular weight membrane protein modulates virus release. J Virol. 65:4107-4113.
Loimas S, Toppinen M R, Visakorpi T, Janne J, Wahlfors J. Human prostate carcinoma cells as targets for herpes simplex virus thymidine kinase-mediated suicide gene therapy. Cancer Gene Ther. 2001 February;8(2):137-44.
Markert J M, Medlock M D, Rabkin S D, Gillespie G Y, Todo T, Hunter W D, Palmer C A, Feigenbaum F, Tomatore C, Tufaro F, Martuza R L. Conditionally replicating herpes simplex virus mutant, G207 for the treatment of malignant glioma: results of a phase I trial. Gene Ther. 2000 May;7(10):867-74.
Mathiot C C, Grimaud G, Garry P, Bouquety J C, Mada A, Daguisy A M, Georges A J. An oubreak of human Semliki Forest virus infections in Central African Republic. Am J Trop Med Hyg. 1990 April;42(4):386-93.
Melani C, Chiodoni C, Arienti F, Maccalli C, Sule-Suso J, Anichini A, Colombo M P, Parmiani G. Cytokine gene transduction in tumor cells: interleukin (IL)-2 or IL-4 gene transfer in human melanoma cells. Nat Immun. 1994 March-June; 13(2-3):76-84.
Okada H, Lieberman F S, Edington H D, Witham T F, Wargo M J, Cai Q, Elder E H, Whiteside T L, Schold S C Jr, Pollack I F. Autologous glioma cell vaccine admixed with interleukin-4 gene transfected fibroblasts in the treatment of recurrent glioblastoma: preliminary observations in a patient with a favorable response to therapy. J Neurooncol. 2003, 64:13-20.
Ramont L, Pasco S, Hornebeck W, Maquart F X, Monboisse J C. Transforming growth factor-betal inhibits tumor growth in a mouse melanoma model by down-regulating the plasminogen activation system. Exp Cell Res. 2003 Nov. 15;291:1-10.
Sauthoff H, Hu J, Maca C, Goldman M, Heitner S, Yee H, Pipiya T, Rom W N, Hay J G. Intratumoral spread of wild-type adenovirus is limited after local injection of human xenograft tumors: virus persists and spreads systemically at late time points. Hum Gene Ther. 2003 Mar. 20;14(5):425-33.
Shah A C, Benos D, Gillespie G Y, Markert J M. Oncolytic viruses: clinical applications as vectors for the treatment of malignant gliomas. J Neurooncol. 2003 December;65(3):203-26.
Spurbeck W, Ng C, Vanin E and Davidoff A. 2003. Retroviral vector-producer cell-mediated in vivo gene transfer of TIMP-3 restricts angiogenesis and neuroblastoma growth in mice. Cancer Gene Therapy 10(3), 161-167.
Takahashi M, Fukami S, Iwata N, Inoue K, Itohara S, Itoh H, Haraoka J, Saido T. In vivo glioma growth requires host-derived matrix metalloproteinase 2 for maintenance of angioarchitecture. Pharmacol Res. 2002 August;46(2):155-63.
Tuittila, M., Santagati, M., Määttä, J. A., Roytta, M. & Hinkkanen, A. E. 2000. Replicase complex genes of Semliki Forest virus confer lethal neurovirulence. J. Virol., 74: 4579-4589.
Tuittila, M. and Hinkkanen, A E. 2003. Amino acid mutations in the replicase protein nsP3 of Semliki Forest virus cumulatively affect neurovirulence. J. Gen. Virol. 84: 1525-1533.
Vähä-Koskela M J, Tuittila M T, Nygardas P T, Nyman J K, Ehrengruber M U, Renggli M, Hinkkanen A E. 2003. A novel neurotropic expression vector based on the avirulent A7(74) strain of Semliki Forest virus. J. Neurovirol. 9: 1-15.
Wick M R, Graeme-Cook F M. Pancreatic neuroendocrine neoplasms: a current summary of diagnostic, prognostic, and differential diagnostic information. Am J Clin Pathol. 2001 June; 115 Suppl: S28-45.
Yaeger M J, Koestner A, Marushige K, Marushige Y. The use of nerve growth factor as a reverse transforming agent for the treatment of neurogenic tumors: in vivo results. Acta Neuropathol (Berl). 1992;83(6):624-9.

1. A method for the treatment of a central nervous system cancer in an individual, said method comprising peripheral administration of a replication competent alphavirus of the Semliki Forest family, or its recombinant, to said individual.

2. The method according to claim 1 wherein the reproduction of the said alphavirus is very limited or it does not reproduce at all in the normal central nervous system cells of an adult individual.

3. The method according to claim 1 wherein the alphavirus is the Semliki Forest A774 strain.

4. The method according to claim 1 wherein the peripheral administration is intraperitoneal, intravenous, intra-arterial, intramuscular, intradermal, subcutaneous, or intranasal administration.

5. The method according to claim 1 wherein the cancer is a brain tumor.

6. The method according to claim 5 wherein the cancer is a glioma.

7. A method for the treatment of a central nervous system cancer in an individual, said method comprising a combination of a surgical operation of a tumor in the central nervous system and administration of a replication competent alphavirus of the Semliki Forest family, or its recombinant, to said individual.

8. The method according to claim 7 wherein the reproduction of the said alphavirus is very limited or it does not reproduce at all in the normal central nervous system cells of an adult individual.

9. The method according to claim 7 wherein the alphavirus is the Semliki Forest A774 strain.

10. The method according to claim 7 wherein said alphavirus or its recombinant is administered intratumorally or intracranially.

11. The method according to claim 7 wherein said alphavirus or its recombinant is administered peripherally.

12. The method according to claim 11 wherein the peripheral administration is intraperitoneal, intravenous, intra-arterial, intramuscular, intradermal, subcutaneous, or intranasal administration.

13. The method according to claim 11 wherein the administration of the said alphavirus or its recombinant is carried out before the surgical operation, after the operation or during the operation.

14. The method according to claim 7 wherein the cancer is a brain tumor.

15. The method according to claim 14 wherein the cancer is a glioma.

16. A pharmaceutical composition comprising a replication competent alphavirus of the Semliki Forest family, or its recombinant, and a therapeutically suitable carrier.

17. The composition according to claim 16 wherein the alphavirus is the Semliki Forest A774 strain.
